SEQUENCE LISTING (2 sequences)

SEQ ID NO: 1 - DNA, 4414 bp, Homo sapiens
  CDS (44)...(1957)
  sig_peptide (44)...(97)
ccgcgctctctgatcagaggcgaagctcggaggtcctacaggtatggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttagcctttctttggtttcccaagagctacgtatttaatagcaaagaattgactggggcaataacgtttaagccaaaacaatgtttaaaccttttttgggggagtgacaggatggggtatggattctaaaatgccttttcccaaaatgttgaaatatgatgttaaaaaaataagaagaatgcttaatcagatagatattcctattgtgcaatgtaaatattttaaagaattgtgtcagactgtttagtagcagtgattgtcttaatattgtgggtgttaatttttgatactaagcattgaatggctatgtttttaatgtatagtaaatcacgctttttgaaaaagcgaaaaaatcaggtggcttttgcggttcaggaaaattgaatgcaaaccatagcacaggctaattttttgttgtttcttaaataagaaacttttttatttaaaaaactaaaaactagaggtgagaaatttaaactataagcaagaaggcaaaaatagtttggatatgtaaaacatttactttgacataaagttgataaagattttttaataatttagacttcaagcatggctattttatattacactacacactgtgtactgcagttggtatgacccctctaaggagtgtagcaactacagtctaaagctggtttaatgttttggccaatgcacctaaagaaaaacaaactcgttttttacaaagcccttttatacctccccagactccttcaacaattctaaaatgattgtagtaatctgcattattggaatataattgttttatctgaatttttaaacaagtatttgttaatttagaaaactttaaagcgtttgcacagatcaacttaccaggcaccaaaagaagtaaaagcaaaaaagaaaacctttcttcaccaaatcttggttgatgccaaaaaaaaatacatgctaagagaagtagaaatcatagctggttcacactgaccaagatacttaagtgctgcaattgcacgcggagtgagttttttagtgcgtgcagatggtgagagataagatctatagcctctgcagcggaatctgttcacacccaacttggttttgctacataattatccaggaagggaataaggtacaagaagcattttgtaagttgaagcaaatcgaatgaaattaactgggtaatgaaacaaagagttcaagaaataagtttttgtttcacagcctataaccagacacatactcatttttcatgataatgaacagaacatagacagaagaaacaaggttttcagtccccacagataactgaaaattatttaaaccgctaaaagaaactttctttctcactaaatcttttataggatttatttaaaatagcaaaagaagaagtttcatcattttttacttcctctctgagtggactggcctcaaagcaagcattcagaagaaaaagaagcaacctcagtaatttagaaatcattttgcaatcccttaatatcctaaacatcattcatttttgttgttgttgttgttgttgagacagagtctcgctctgtcgccaggctagagtgcggtggcgcgatcttgactcactgcaatctccacctcccacaggttcaggcgattcccgtgcctcagcctcctgagtagctgggactacaggcacgcaccaccatgccaggctaatttttttgtattttagcagagacggggtttcaccatgttggccaggatggtctcgagtctcctgacctcgtgatccacccgactcggcctcccaaagtgctgggattacaggtgtaagccaccgtgcccagccctaaacatcattcttgagagcattgggatatctcctgaaaaggtttatgaaaaagaagaatctcatctcagtgaagaatacttctcattttttaaaaaagcttaaaactttgaagttagctttaacttaaatagtatttcccatttatcgcagaccttttttaggaagcaagcttaatggctgataattttaaattctctctcttgcaggaaggactatgaaaagctagaattgagtgtttaaagttcaacatgttatttgtaatagatgtttgatagattttctgctactttgctgctatggttttctccaagagctacataatttagtttcatataaagtatcatcagtgtagaacctaattcaattcaaagctgtgtgtttggaagactatcttactatttcacaacagcctgacaacatttctatagccaaaaatagctaaatacctcaatcagtctcagaatgtcattttggtactttggtggccacataagccattattcactagtatgactagttgtgtctggcagtttatatttaactctctttatgtctgtggattttttccttcaaagtttaataaatttattttcttggattcctgataatgtgcttctgttatcaaacaccaacataaaaatgatctaaacc

SEQ ID NO: 2 - protein, 638 aa, Homo sapiens
  SIGNAL (1)...(18)
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

(19) United States
(12) Patent Application Publication
(10) Pub. No.: US 2006/0188945 A1
(43) Pub. Date: Aug. 24, 2006

(54) Screening methods

(76) Inventors: Vendela Parrow, Uppsala (SE); Lars Olof Bergquist, Tyreso (SE); Catrine Dreifeldt, Bromma (SE); Christine Flodin, Vallentuna (SE)

Correspondence Address: FISH & RICHARDSON PC, P.O. BOX 1022, MINNEAPOLIS, MN, 55440-1022, US

(21) Appl. No.: 11/314,257

(22) Filed: Dec. 21, 2005

Related U.S. Application Data
(60) Provisional application No. 60/638,694, filed on Dec. 23, 2004.

(51) Int. Cl. G01N 33/567; G01N 33/53

(52) U.S. Cl. 435/7.2

(57) ABSTRACT

The present invention relates to methods for the identification of modulators of cytokine class I receptors by determining whether a compound that binds to a cytokine class I receptor at a site different from the binding site of the naturally-occurring cytokine ligand is effective at modulating the amount of the cytokine class I receptor on the surface of the cell.

### CROSS REFERENCE TO RELATED APPLICATIONS

This application claims priority from U.S. Provisional Patent Application No. 60/638,694, filed Dec. 23, 2004. The prior application is incorporated herein by reference in its entirety.

### TECHNICAL FIELD

The present invention relates to methods for identifying agents that modulate the activity of cytokine class I receptors, such as the growth hormone receptor. The agents are useful for the treatment or prevention of medical disorders caused by cytokine or cytokine receptor dysregulation.

### BACKGROUND

Growth hormone (GH) is secreted from the adenohypophysis (anterior pituitary gland) and has a variety of target tissues. GH has a range of actions including somatic growth, differentiation, and intermediary metabolism, effects that are mediated by GH-induced insulin-like growth factor-1 (IGF-1) (Bichell et al. (1992) Mol. Endocrin. 6: 1899-1908). IGF-1 is the major regulator of postnatal body growth, and has both endocrine and paracrine action on different tissues. Several intracellular second messengers have been implicated in the signal transduction of GH, including calcium ions, phospholipase C, phospholipase A2, G-proteins, protein kinase C (PKC), Janus kinase 2 (JAK2) and signal transducer and activator of transcription (STAT) 1, 3 and 5.

GH induces transcription of different genes by binding to the growth hormone receptor (GHR), a membrane-associated receptor which belongs to the superfamily of cytokine (class I) receptors (Graichen et al. (2003) J. Biol. Chem. 278: 6346-6354). In addition to GHR, the cytokine class I receptor superfamily includes receptors for prolactin, erythropoietin, granulocyte colony-stimulating factor, granulocyte-macrophage colony stimulating factor, ciliary neutrophic factor, thrombopoietin, leptin, cardiotrophin I, and the β-chain of interleukin (IL)-2 through IL-7, IL-9, and IL-11 to IL-13 (Cosman, D. et al. (1990) Trends Biochem. Sci. 15: 265-270; see also Taga, T. & Kishimoto, T.: Signal transduction through class I cytokine receptors; pp. 19-36 in: Signal Transduction. Eds. Heldin, C.-H. and Purton, M., Chapman Hall, 1996). The cytokine class I receptors lack intrinsic catalytic activity, but are associated to cytosolic proteins having tyrosine-kinase activity. Cytokine class I receptors possess a single membrane-spanning domain and exist as monomers that dimerize and become activated upon ligand binding.

To regulate the number of GH receptors on the cell surface, GHR is internalized in the cell by endocytosis. Receptor internalization is part of the signal transduction mechanism, and has also been described for the insulin receptor (Podlecki et al. (1987) J. Biol. Chem. 262: 3362-3368), epidermal growth factor receptor (Jiang and Schindler (1990) J. Cell. Biol. 110: 559-568), and prolactin receptor (Juu-Chin Lu et al. (2002) Molecular Endocrinology 16:2515-2527).

Receptor internalization has been established as a part of the down-regulation of the stimulatory action of a hormone (Van Kerkhof, P et al. (2000) J. Biol. Chem. 275: 1575-1580). After endocytosis the ligand-receptor complex is degraded in the lysosomes. Alternatively, the hormone becomes degraded and the receptor re-circulated to the cell membrane.

GHR has been reported to translocate to the nucleus upon GH-stimulation (Lobie et al. (1994) J. Biol. Chem. 269: 31735-31746) and GH and GHR may be translocated to the nucleus in association (Lobie et al. (1994) J. Biol. Chem. 269: 21330-21339). The nuclear translocation of GH and GHR is independent of JAK2 (Graichen, R. et al. (2003) J. Biol. Chem. 278: 6346-6354), which suggests that nuclear translocation may be an alternative signal transduction pathway independent of the JAK-STAT pathway. The extracellular part of GHR (GHBP) has also been reported to translocate to the nucleus where it enhances GH-induced STAT5-activated transcription as well as STAT5-activated transcription mediated by other members of the cytokine receptor superfamily (Graichen et al., supra), indicating that the nuclear GHBP is functional.

It appears possible that GHR internalization has more than one function, namely as a means for down-regulation and clearance. Consequently, there is a need to develop methods for studying the amounts of membrane bound receptor and the subcellular distribution (endoplasmatic vesicles, ER, nuclear) of GHR after stimulation with ligands or compounds binding to the ligand-binding site, as well as after stimulation with substances binding to other parts of GHR.

### BRIEF DESCRIPTION OF THE DRAWINGS

FIGS. 1A and 1B are graphs depicting the amount of growth hormone receptor present on a human liver cell line (1A) and a correlation of the amount of DNA to the number of cells in the plate (1B).

FIG. 2 is a graph depicting the amount of membrane bound growth hormone receptor present on a human osteosarcoma cell line after treatment with various concentrations of BVT.3693.

### DETAILED DISCLOSURE

The present invention is based, at least in part, on the discovery that a compound that binds to a cytokine class I receptor at a site other than the cytokine-binding site can induce receptor internalization and/or shedding in the absence of the endogenous cytokine ligand. This unexpected finding identifies regions outside of the ligand-binding site as important targets for receptor antagonists and/or agonists and reveals that the presence of a cytokine class I receptor on the cell surface can be modulated by the binding of such compounds (i.e., in the absence of ligands binding to the ligand-binding site of the receptor). In light of these findings, the detection of internalization, subcellular distribution, and/or shedding of membrane bound receptors by such compounds can be used as means to identify pharmacologically active compounds.

The present invention provides a method of characterizing the bioactivity of a compound that binds to a cytokine class I receptor. The method includes the following steps: (1) providing a cell expressing a cytokine class I receptor (e.g., growth hormone receptor) on its cell surface; (2) contacting the cell with a compound that binds to the cytokine class I receptor at a site different from the binding site of the naturally-occurring cytokine ligand; and (3) determining whether the compound modulates the amount of the cytokine class I receptor on the surface of the cell.

The invention also provides a method of identifying a modulator of a cytokine class I receptor. The method includes the following steps: (1) screening to identify a compound that binds to a cytokine class I receptor (e.g., growth hormone receptor) at a site different from the binding site of the naturally-occurring cytokine ligand; (2) contacting a cell expressing the cytokine class I receptor on its cell surface with the compound; and (3) determining whether the compound modulates the amount of the cytokine class I receptor on the surface of the cell.

The family of “cytokine class I receptors” includes, for example, receptors for growth hormone, prolactin, erythropoietin, granulocyte colony-stimulating factor, granulocyte-macrophage colony stimulating factor, ciliary neutrophic factor, thrombopoietin, leptin, cardiotrophin I, and the β-chain of interleukin (IL)-2 through IL-7, IL-9, and IL-11 to IL-13. Experiments using the growth hormone receptor are detailed herein in Examples 1 and 2. As a result of structural features shared between the growth hormone receptor and other members of this family, the methods described herein are expected to be generally effective on members of the cytokine class I receptor family.

BVT.3693 (N-[5-(aminosulfonyl)-2-methylphenyl]-5-bromo-2-furamide) is an exemplary compound described herein that binds to the growth hormone receptor at a site different from the binding site of growth hormone. Compounds that bind outside of a receptor's ligand-binding site can be identified using routine methods. For example, the binding ability of a candidate compound can be evaluated using a mutant or fragment of a cytokine class I receptor that lacks the ability to bind the endogenous ligand (e.g., identify a compound that can bind to such a mutant or fragment). In addition, a compound can be analyzed for its ability to bind the receptor when the ligand is bound to the receptor (indicating that the compound binds outside of the ligand binding site). Alternatively, a compound that has previously been characterized to have the desired binding property (such as BVT.3693) can be used in a competitive inhibitor assay to identify alternative compounds that bind to a receptor outside of the binding site of the endogenous ligand.

Many of the methods described herein are cell-based screens that use cells expressing a cytokine class I receptor on the cell surface. Cells that can be used in such methods include (1) primary cells or cell lines that naturally express the receptor on the surface, (2) cells into which an expression vector expressing the receptor has been introduced (e.g., cells transfected with a plasmid encoding the receptor or infected with a virus encoding the receptor), or (3) cells that have been contacted with a molecule that induces expression of the receptor. The cDNA sequences of cytokine class I receptors are well known. The cDNA sequence of the growth hormone receptor, which is analyzed in detail in the Examples, is depicted in SEQ ID NO:1 (the predicted amino acid sequence is depicted in SEQ ID NO:2).

The methods described herein can include steps that analyze one or more of a variety of biochemical events that may result from the modulation of the cytokine class I receptor on the cell surface. For example, the method can include a step of determining whether the compound induces internalization of the cytokine class I receptor. In another example, the method can include a step of determining whether the compound induces shedding of the cytokine class I receptor.

A determination of whether a compound modulates the amount of a cytokine class I receptor on the surface of a cell can be carried out by a variety of methods. For example, immunofluorescence can be used to determine whether the receptor is present on the surface of a cell (e.g., before or after the cell is contacted with a test compound) and/or whether the receptor has been translocated to a particular compartment of a cell. Antibodies that specifically recognize the receptor can be used in such analyses. In addition, the receptor itself can be engineered as a fusion protein that contains the receptor fused to a fluorescent label (e.g., green fluorescent protein). In such circumstances, the cellular localization of the receptor fusion protein can be tracked without the need for use of antibodies or other agents that bind to the receptor. In addition to immunofluorescence, the presence or amount of a receptor on the cell surface can be evaluated by contacting a cell with a labeled antibody or labeled ligand and determining whether the antibody or ligand binds to the receptor on the cell surface.

In one embodiment, the method includes a step of evaluating the subcellular distribution of the cytokine class I receptor following the contacting of the cell with the compound. Such an analysis can include (i) determining whether the cytokine class I receptor is translocated to the nucleus following the contacting of the cell with the compound, (ii) determining whether the cytokine class I receptor is translocated to the cytoplasm following the contacting of the cell with the compound, or (iii) determining whether the cytokine class I receptor is translocated to the nucleus, the cytoplasm, or the nucleus and cytoplasm following the contacting of the cell with the compound.

In some embodiments, the methods compare properties of the cytokine ligand to the compound that binds to the cytokine class I receptor at a site different from the cytokine ligand. In one example, the method includes comparing the amount of the cytokine class I receptor translocated to the nucleus following the contacting of the cell with the compound to the amount of the cytokine class I receptor translocated to the nucleus following contacting the cell with the cytokine. In another example, the method includes comparing the kinetics of internalization of the cytokine class I receptor following the contacting of the cell with the compound to the kinetics of internalization of the cytokine class I receptor following contacting the cell with the cytokine.

In some embodiments, the methods include steps of: (1) comparing receptor internalization induced by contacting the cell with the compound to receptor internalization induced by contacting the cell with the cytokine; and (2) selecting the compound as a candidate pharmaceutical agent if the compound induces receptor internalization at a level or a rate that is equal to or exceeds the level or rate of receptor internalization induced by the cytokine.

In some embodiments, the methods include steps of: (1) comparing the subcellular distribution of the cytokine class I receptor induced by contacting the cell with the compound to the subcellular distribution of the cytokine class I receptor induced by contacting the cell with the cytokine; and (2) selecting the compound as a candidate pharmaceutical agent if the compound induces receptor internalization but results in a subcellular distribution of the cytokine class I receptor that differs from that induced by the cytokine.

In some embodiments, the methods include steps of: (1) comparing the nuclear translocation of the cytokine class I receptor induced by contacting the cell with the compound to the nuclear translocation of the cytokine class I receptor induced by contacting the cell with the cytokine; and (2) selecting the compound as a candidate pharmaceutical agent if the compound induces receptor internalization but results in decreased nuclear translocation of the cytokine class I receptor as compared to that induced by the cytokine.

In some embodiments, the methods include steps of: (1) comparing the cytoplasmic translocation of the cytokine class I receptor induced by contacting the cell with the compound to the cytoplasmic translocation of the cytokine class I receptor induced by contacting the cell with the cytokine; and (2) selecting the compound as a candidate pharmaceutical agent if the compound induces receptor internalization but results in increased cytoplasmic translocation of the cytokine class I receptor as compared to that induced by the cytokine.

Compounds characterized and/or identified according to the methods described herein can be useful in prophylaxis and/or therapy. For example, a compound that binds to the growth hormone receptor can be used for treating or preventing acromegaly, cancer, diabetes, diabetic nephropathy, diabetic retinopathy and neuropathy, and other diseases with pathologically increased IGF-I levels, as well as for treatment of children with growth hormone deficiency, Prader-Willis syndrome, Turners syndrome, children with retarded growth due to chronic renal failure, substitution of adults with growth hormone deficiency, frail elderly, and wasting syndrome in AIDS.

The present invention is also based, at least in part, on the discovery that certain fluorescent DNA stains can be used effectively to determine the number of cells present in a cell sample. In addition, a particular fluorescent DNA stain was found to have surprisingly advantageous properties in such detection methods.

Accordingly, in another aspect, the invention provides a method for determining the number of cells in a cell sample. The method includes the following steps: (1) providing a cell sample immobilized on a solid surface; (2) contacting the cell sample with a fluorescent DNA stain (e.g., Vistra Green); (3) incubating the cell sample in the presence of the fluorescent DNA stain; (4) measuring the amount of fluorescence emitted by the cell sample; and (3) comparing the measured fluorescence to a standard curve to determine the number of cells present in the cell sample. The fluorescent DNA stain can be, for example, Vistra Green or ethidium bromide.

In some embodiments, the cell sample is not washed between the steps of contacting with the fluorescent DNA stain and measuring the amount of fluorescence emitted by the cell sample.

These method can include the additional steps of, prior to contacting the cell sample with the fluorescent DNA stain, determining the amount of a protein in the cell sample immobilized on the solid surface. The protein can be a cell surface receptor, e.g., a cytokine class I receptor such as the growth hormone receptor. In embodiments where the protein is a cell surface receptor, the method can include determining the amount of the cell surface receptor present on the surface of the cell. In such embodiments, the method serves as internal standard to identify the number of cells present on the solid surface (e.g., a well of a microplate).

The methods allow for an accurate correlation between the amount of a protein present in a sample and the actual number of cells in the sample. The use of the fluorescent DNA stain (e.g., Vistra Green) is advantageous over other methods because it does not require additional handling (such as washing) of the cell sample after the detection of the protein. Such additional handling can result in sample loss and a distortion of the data. Accordingly, addition of the fluorescent DNA stain directly to a well of a microplate permits a correct determination of the amount of DNA present in proportion to the amount of protein detected.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention will now be further illustrated through the description of examples of its practice. The examples are not intended as limiting in any way of the scope of the invention.

### EXAMPLES

### Example 1

### Subcellular Distribution of Growth Hormone Receptor in the Presence or Absence of Growth Hormone and BVT.3693

WRL-68 cells were cultured in EMEM medium with NaHCO₃ (Statens Veterinärmedicinska Anstalt, Uppsala, Sweden), supplemented with 10% fetal bovine serum (FBS), 2% L-Glutamine, 1% Pyruvate, and non-essential acids (NEA), all from GIBCO, at 37° C. in 5% CO₂.

Transfection: Cells were transfected with plasmid DNA encoding the full-length human GHR (pMB 1288, 2 μg/μl) using DOTAP Liposomal Transfection Reagent (Roche), according to the manufacturers instructions. WRL-68 cells in T75 flasks were transfected with 10 μg DNA for each flask. 5 μl (10 μg) DNA was diluted with 250 μl OPTIMEM1 medium (GIBCO), and mixed with 75 μl DOTAP reagent diluted in 175 μl OPTIMEM1 medium. The mixture was incubated for 10 minutes and then mixed with 10 ml OPTIMEM1 medium. Cells were washed once and then incubated for 4 hours with the DNA/DOTAP transfection mix. The transfection reagents were removed and fresh culture medium was added. Cells were re-seeded into new culture dishes the following day.

Stimulation: 3×10⁴ cells were seeded on chamber slides in eight-well dishes and grown for two days. After starvation of fetal calf serum for 15 minutes to 12 hours, the cells were stimulated at different time intervals, 5 to 130 minutes, with a final concentration of 10-100 nM GH (Pharmacia) or 2 μM BVT.3693 (N-[5-(aminosulfonyl)-2-methylphenyl]-5-bromo-2-furamide). After treatment, the cells were rinsed twice with ice-cold PBS. Fixation was performed with 4% paraformaldehyde for 20 minutes.

Staining with rabbit antiserum: The chamber slides were washed twice by immersion of the slides in a container with 0.05% TBS-Tween. Cells were permeabilized with 0.1% Triton X-100 (Sigma) for 5 minutes and then washed as described above. The cells were blocked with 10% FBS for 10 minutes, washed two times, and incubated with rabbit antiserum diluted 1:100 (Agrisera) at 4° C. overnight. The slides were washed three times and then blocked with 0.1% FBS in 30 minutes. Finally, cells were incubated with TRITC conjugated goat anti-rabbit antibody diluted 1:50 (Immunotech) for 30 minutes and washed three times. The cover slips were mounted with Slow Fade Light Antifade (Molecular Probes). Controls were performed in two different ways, by omission of antiserum or by replacement with the preimmune rabbit serum. All dilutions were made in TBS-Tween.

Staining with Mab 263: The chamber slides were washed twice by immersion of the slides in a container with 0.05% TBS-Tween. Cells were permeabilized with 0.1% Triton X-100 (Sigma) for 5 minutes and then washed as described above. The cells were blocked with 10% FBS for 10 minutes, washed two times and incubated with Mab 263 (Agen Biomedical LTD) 1:100 at 4° C. overnight. The slides were washed three times and then blocked with 0.1% FBS for 30 minutes. Finally, cells were incubated with FITC conjugated rabbit anti-mouse antibody diluted 1:20 (Dako) for 30 minutes and washed three times. The cover slips were mounted with Slow Fade Light Antifade (Molecular Probes). Controls were performed by omission of Mab 263. All dilutions were made in TBS-Tween.

Alterations in the subcellular distribution of GHR were detected by immunofluorescence using Mab263 (mouse anti-GHR antibody) after stimulation with GH or the GHR-binding compound, BVT.3693. 8-well chamber slides were used to culture the cells, and parallel slides were treated with either Triton-X 100 (to visualise the amount of receptor “inside” the cells) or Tween (to visualise the receptor “outside” on the membrane). In unstimulated cells, most of the receptor can be found on, or near the plasma membrane. In cells stimulated with GH, the receptor becomes localized to the nucleus. Cells treated with the compound BVT.3693 also showed an intracellular staining, with both a nuclear localisation and a cytoplasmic localisation of the receptor.

Internalization of GHR was seen within 5 to 60 minutes after stimulation with 10 nM GH. In contrast, receptor internalization was seen within 20 to 130 minutes after stimulation with 2 μM BVT.3693. These results demonstrated that both the subcellular distribution of GHR and the kinetics of receptor internalization differ after treatment with BVT.3693 as compared to GH. Thus, a compound that, like BVT.3693, binds to GHR at a site other than the ligand-binding site is capable of altering the subcellular distribution of GHR.

### Example 2

### DELFIA Assay for Determination of Receptor on Cell Surface

The following method was used to quantify the number of GH receptors on the cell surface after stimulation with different compounds.

On day 1, WRL-68 cells were seeded at a density of 15,000 cells per well and incubated under standard cell culture conditions.

On day 3, cells were washed with serum-free medium and incubated 1 hour prior to stimulation with a dose-response of BVT.3693. The cells were then washed with ice-cold PBS (Dulbecco PBS-A) and fixated with 4% paraformaldehyde on ice for 20 minutes. Thereafter the plates were washed with PBS and stored in a refrigerator until used. GHR antibody (diluted 1/100-1/500) was added to the wells and the plates were incubated overnight in a refrigerator.

On day 4, the plates were washed with PBS using a DELFIA plate washer. A secondary biotin conjugated antibody was then added and the plates were incubated at room temperature for 30 minutes. The plates were washed again with a plate washer and Streptavidin-Europium was added. The plates were incubated for 30 minutes, followed by three more washes in DELFIA wash-buffer, then DELFIA enhancement solution was added (both from Wallac). The amount of Europium fluorescence was determined by reading in a Victor analysis instrument.

To correlate the amount of Europium fluorescence measured (which fluorescence is proportional to the number of GH receptors present on the cell surface) to the number of cells attached to the bottom of the well after all the washing steps, we developed an internal standard. In developing this standard, we required that the method not involve any additional washing steps. Thus the detection method for the internal standard must be compatible with the DELFIA enhancement buffer present in the plate after the Europium measurement. For this reason, standard protein concentration measurements could not be used. Instead, we developed a method to quantify the amount of DNA in the wells as an internal standard.

Two different approaches were tried: using Hoechst bisbenzimide, as previously described in the literature; or using Vistra Green™ (Amersham Biosciences, Sunnyvale, Calif.) a fluorophor previously used to stain agarose gels after gel-electrophoresis. We found that Vistra Green gave a much better correlation to the number of cells. In addition, the Vistra Green stain could be added directly to the Europium Enhancement solution, thus obviating the need for extra washing steps after quantifying the Europium. The plates were incubated on a plate shaker for 30 minutes, and the amount of Vistra Green fluorescence was determined. The number of cells in each well was then calculated from a standard curve. The amount of receptor present on the cell surface, as measured by the Europium luminescence, could thus be correlated to the number of cells present in each well.

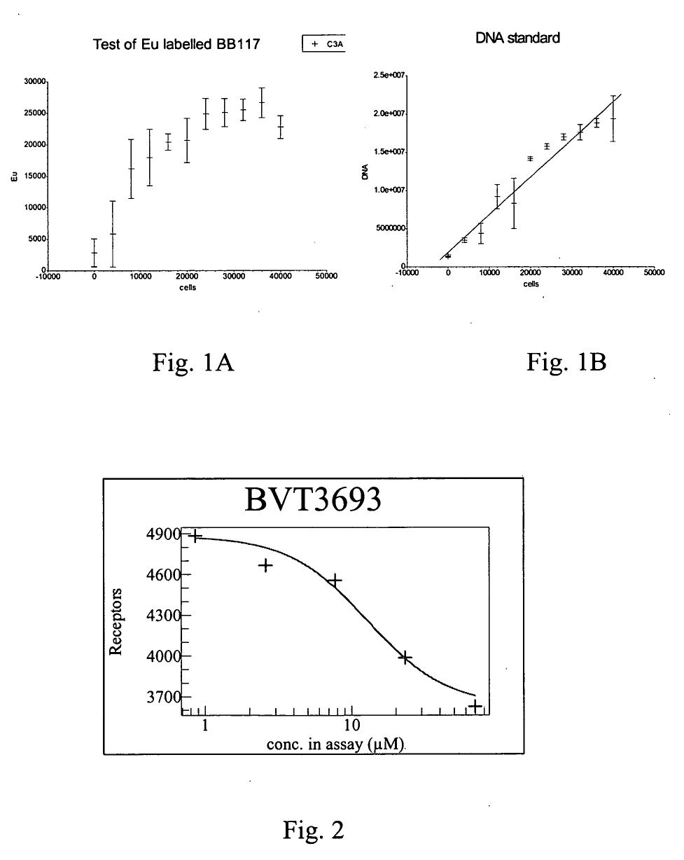

FIGS. 1A and 1B show the results from an assay detecting: the amount of GHR on the surface of the human liver cell-line C3A using Eu-labeled rabbit anti GH-rec antibody BB117 (FIG. 1A); and the amount of DNA correlating to the number of cells on the same plate (FIG. 1B).

FIG. 2 shows the results from an assay measuring the effect of the compound BVT.3693 on the amount of membrane bound GHR on a human osteosarcoma cell line. 10,000 cells per well were seeded the day before stimulation. The cells were stimulated for 1.5 hours with increasing concentrations of BVT.3693. Receptor quantification was performed using an Eu-labeled anti GH-rec antibody BB117, as described above (simplified protocol). Consistent with the immunofluorescence findings presented in Example 1, BVT.3693 induced a dose dependent reduction in the amount of GHR on the surface of the cells.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method of characterizing the bioactivity of a compound that binds to a cytokine class I receptor, the method comprising:
providing a cell expressing a cytokine class I receptor on its cell surface;
contacting the cell with a compound that binds to the cytokine class I receptor at a site different from the binding site of the naturally-occurring cytokine ligand; and
determining whether the compound modulates the amount of the cytokine class I receptor on the surface of the cell.

2. The method of claim 1, wherein the method comprises determining whether the compound induces internalization of the cytokine class I receptor.

3. The method of claim 1, wherein the method comprises determining whether the compound induces shedding of the cytokine class I receptor.

4. The method of claim 1, further comprising evaluating the subcellular distribution of the cytokine class I receptor following the contacting of the cell with the compound.

5. The method of claim 1, further comprising determining whether the cytokine class I receptor is translocated to the nucleus following the contacting of the cell with the compound.

6. The method of claim 1, further comprising determining whether the cytokine class I receptor is translocated to the cytoplasm following the contacting of the cell with the compound.

7. The method of claim 1, further comprising determining whether the cytokine class I receptor is translocated to the nucleus, the cytoplasm, or the nucleus and cytoplasm following the contacting of the cell with the compound.

8. The method of claim 1, further comprising comparing the amount of the cytokine class I receptor translocated to the nucleus following the contacting of the cell with the compound to the amount of the cytokine class I receptor translocated to the nucleus following contacting the cell with the cytokine.

9. The method of claim 2, further comprising comparing the kinetics of internalization of the cytokine class I receptor following the contacting of the cell with the compound to the kinetics of internalization of the cytokine class I receptor following contacting the cell with the cytokine.

10. The method of claim 2, wherein the method comprises:
comparing receptor internalization induced by contacting the cell with the compound to receptor internalization induced by contacting the cell with the cytokine; and
selecting the compound as a candidate pharmaceutical agent if the compound induces receptor internalization at a level or a rate that is equal to or exceeds the level or rate of receptor internalization induced by the cytokine.

11. The method of claim 1, wherein the method comprises:
comparing the subcellular distribution of the cytokine class I receptor induced by contacting the cell with the compound to the subcellular distribution of the cytokine class I receptor induced by contacting the cell with the cytokine; and
selecting the compound as a candidate pharmaceutical agent if the compound induces receptor internalization but results in a subcellular distribution of the cytokine class I receptor that differs from that induced by the cytokine.

12. The method of claim 1, wherein the method comprises:
comparing the nuclear translocation of the cytokine class I receptor induced by contacting the cell with the compound to the nuclear translocation of the cytokine class I receptor induced by contacting the cell with the cytokine; and
selecting the compound as a candidate pharmaceutical agent if the compound induces receptor internalization but results in decreased nuclear translocation of the cytokine class I receptor as compared to that induced by the cytokine.

13. The method of claim 1, wherein the method comprises:
comparing the cytoplasmic translocation of the cytokine class I receptor induced by contacting the cell with the compound to the cytoplasmic translocation of the cytokine class I receptor induced by contacting the cell with the cytokine; and
selecting the compound as a candidate pharmaceutical agent if the compound induces receptor internalization but results in increased cytoplasmic translocation of the cytokine class I receptor as compared to that induced by the cytokine.

14. The method of claim 1, wherein the cytokine is growth hormone and the cytokine class I receptor is the growth hormone receptor.

15. A method of identifying a modulator of a cytokine class I receptor, the method comprising:
screening to identify a compound that binds to a cytokine class I receptor at a site different from the binding site of the naturally-occurring cytokine ligand;
contacting a cell expressing the cytokine class I receptor on its cell surface with the compound; and
determining whether the compound modulates the amount of the cytokine class I receptor on the surface of the cell.

16. The method of claim 15, wherein the method comprises determining whether the compound induces internalization of the cytokine class I receptor.

17. The method of claim 15, wherein the method comprises determining whether the compound induces shedding of the cytokine class I receptor.

18. The method of claim 15, further comprising evaluating the subcellular distribution of the cytokine class I receptor following the contacting of the cell with the compound.

19. The method of claim 15, further comprising determining whether the cytokine class I receptor is translocated to the nucleus following the contacting of the cell with the compound.

20. The method of claim 15, further comprising determining whether the cytokine class I receptor is translocated to the cytoplasm following the contacting of the cell with the compound.

21. The method of claim 15, further comprising determining whether the cytokine class I receptor is translocated to the nucleus, the cytoplasm, or the nucleus and cytoplasm following the contacting of the cell with the compound.

22. The method of claim 15, further comprising comparing the amount of the cytokine class I receptor translocated to the nucleus following the contacting of the cell with the compound to the amount of the cytokine class I receptor translocated to the nucleus following contacting the cell with the cytokine.

23. The method of claim 15, further comprising comparing the kinetics of internalization of the cytokine class I receptor following the contacting of the cell with the compound to the kinetics of internalization of the cytokine class I receptor following contacting the cell with the cytokine.

24. The method of claim 15, wherein the method comprises:
comparing receptor internalization induced by contacting the cell with the compound to receptor internalization induced by contacting the cell with the cytokine; and
selecting the compound as a candidate pharmaceutical agent if the compound induces receptor internalization at a level or a rate that is equal to or exceeds the level or rate of receptor internalization induced by the cytokine.

25. The method of claim 15, wherein the method comprises:
comparing the subcellular distribution of the cytokine class I receptor induced by contacting the cell with the compound to the subcellular distribution of the cytokine class I receptor induced by contacting the cell with the cytokine; and
selecting the compound as a candidate pharmaceutical agent if the compound induces receptor internalization but results in a subcellular distribution of the cytokine class I receptor that differs from that induced by the cytokine.

26. The method of claim 15, wherein the method comprises:
comparing the nuclear translocation of the cytokine class I receptor induced by contacting the cell with the compound to the nuclear translocation of the cytokine class I receptor induced by contacting the cell with the cytokine; and
selecting the compound as a candidate pharmaceutical agent if the compound induces receptor internalization but results in decreased nuclear translocation of the cytokine class I receptor as compared to that induced by the cytokine.

27. The method of claim 15, wherein the method comprises:
comparing the cytoplasmic translocation of the cytokine class I receptor induced by contacting the cell with the compound to the cytoplasmic translocation of the cytokine class I receptor induced by contacting the cell with the cytokine; and
selecting the compound as a candidate pharmaceutical agent if the compound induces receptor internalization but results in increased cytoplasmic translocation of the cytokine class I receptor as compared to that induced by the cytokine.

28. The method of claim 15, wherein the cytokine is growth hormone and the cytokine class I receptor is the growth hormone receptor.

29. A method for determining the number of cells in a cell sample, the method comprising:
providing a cell sample immobilized on a solid surface;
contacting the cell sample with a fluorescent DNA stain comprising Vistra Green;
incubating the cell sample in the presence of the fluorescent DNA stain;
measuring the amount of fluorescence emitted by the cell sample; and
comparing the measured fluorescence to a standard curve to determine the number of cells present in the cell sample.

30. The method of claim 29, wherein the cell sample is not washed between the steps of contacting with the fluorescent DNA stain and measuring the amount of fluorescence emitted by the cell sample.

31. The method of claim 29, further comprising, prior to contacting the cell sample with the fluorescent DNA stain, determining the amount of a protein in the cell sample immobilized on the solid surface.

32. The method of claim 31, wherein the protein is a cell surface receptor.

33. The method of claim 32, wherein the method comprises determining the amount of the cell surface receptor present on the surface of the cell.

34. The method of claim 32, wherein the cell surface receptor is a cytokine class I receptor.

35. The method of claim 34, wherein the cytokine class I receptor is the growth hormone receptor.
